(19) United States
(12) Patent Application Publication
(10) Pub. No.: US 2003/0152597 A1
(43) Pub. Date: Aug. 14, 2003

(54) Use of at least one sapogenin for prevening the skin or ageing symptoms

(76) Inventors: Christel Liviero, Paris (FR); Josiane Allec, Antibes (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/182,730

(22) PCT Filed: Nov. 26, 2001
(86) PCT No.: PCT/FR01/03727

(30) Foreign Application Priority Data
Dec. 11, 2000 (FR) 00 16074

(51) Int. Cl. A61K 31/704; A61K 35/78; A61K 7/06

(52) U.S. Cl. 424/401; 424/773; 514/26

(57) ABSTRACT

The invention relates to the cosmetic use of at least one sapogenin or of a natural extract containing it to prevent the signs of ageing of the skin, in particular the loss of elasticity and/or tonicity of the skin and/or the formation of wrinkles and fine lines, by inhibiting the activity of collagenases.

The invention also relates to the cosmetic use of at least one sapogenin or of a natural extract containing it to prevent the loss of firmness of the skin of the human face and/or neck.

The invention relates to the cosmetic use of at least one sapogenin or of a natural extract containing it to prevent the signs of ageing of the skin, in particular the loss of elasticity and/or tonicity of the skin, by inhibiting the activity of collagenases.

The invention also relates to the cosmetic use of at least one sapogenin or of a natural extract containing it to prevent the loss of firmness of the skin of the human face and/or neck.

Human skin consists of two compartments, namely a surface compartment, the epidermis, and a deep compartment, the dermis.

Natural human epidermis is composed mainly of three types of cells: keratinocytes, which form the great majority, melanocytes and Langerhans cells. Each of these cell types contributes, by virtue of its intrinsic functions, towards the essential role played in the body by the skin.

The dermis gives the epidermis a solid support. It is also the epidermis' nourishing factor. It consists mainly of fibroblasts and of an extracellular matrix which is itself composed mainly of collagen, elastin and a substance known as ground substance, these components being synthesized by the fibroblasts. Leukocytes, mastocytes and tissue macrophages are also found therein. It also contains blood vessels and nerve fibres.

These collagen fibres give the dermis its firmness. Collagen fibres consist of fibrils sealed together, thus forming more than 10 different types of structures. The firmness of the dermis is mainly due to the high-quality entanglement of the collagen fibres packed together in all directions. The collagen fibres contribute towards the firmness, elasticity and tonicity of the skin and/or mucous membranes.

The collagen fibres are under constant renewal, but this renewal decreases with age, leading to thinning of the dermis. In addition, various factors lead to the degradation of collagen, with all the consequences which may be envisaged on the structure and/or firmness of the skin and/or mucous membranes.

Specifically, although very strong, collagen fibres are sensitive to certain enzymes known as collagenases. A degradation of collagen fibres leads to the development of flaccid and wrinkled skin, which people, preferring the appearance of smooth and taut skin, have always sought to combat.

Collagenases form part of a family of enzymes known as metalloproteases (MMPs) which are themselves members of a family of proteolytic enzymes (endoproteases) which contain a zinc atom coordinated to 3 cysteine residues and one methionine residue in their active site and which degrade the macromolecular components of the extracellular matrix and of the basal layers at neutral pH (collagen, elastin, etc.).

Prolonged exposure to ultraviolet rays, particularly to ultraviolet rays of A and/or B type, has the effect of stimulating the expression of collagenases, particularly of MMP-1. This is one of the components of photo-induced ageing of the skin.

The importance of collagen in the structure of tissues, particularly the skin and/or mucous membranes, and the importance of combating its degradation in order thus to combat ageing and its consequences is therefore understood by reading the above.

One of the aims of the present invention is thus to be able to provide a product which has an inhibitory effect on collagenases and, if possible, no appreciable side effects.

However, surprisingly and unexpectedly, the Applicant has now discovered that certain natural extracts that are rich in sapogenins, in particular in diosgenin, show inhibitory activity on the activity of collagenases.

Sapogenins are compounds resulting from the acidic hydrolysis of saponosides, which are themselves heterosides of very high molecular weight present in the plant kingdom.

These compounds have in common a steroid structure comprising a variable number of hydroxyl and/or oxo substituents and/or a variable number of double bonds. They are known as natural chemical precursors of steroid hormones and are described, in this respect, as constituents of choice in various cosmetic or pharmaceutical preparations.

Diosgenin has already been described as an anti-inflammatory (Yamada et al., Am. J. Physiol., 273:G355-G364, 1997) and as a slimming active agent due to its action on adipocytes (WO 00/30603). Extracts of Dioscorea opposita and Dioscorea tokora, which contain diosgenin, have moreover been described as being effective for moisturizing the skin and thus making it supple (JP-10 194 947 and JP-2000 143 488).

To the Applicant's knowledge, the inhibitory activity (or inhibitory effect) on collagenase activity of sapogenins and/or natural extracts containing them has, however, never been described.

Thus, one subject of the invention is the cosmetic use of at least one sapogenin or of a natural extract containing it to prevent the signs of ageing of the skin, in particular the loss of elasticity and/or tonicity of the skin and/or the formation of wrinkles and fine lines, by inhibiting the activity of collagenases.

A subject of the invention is also the cosmetic use of at least one sapogenin or of a natural extract containing it to prevent the loss of firmness of the skin of the human face and/or neck.

The sapogenin used according to the invention may be chosen from: diosgenin, hecogenin, smilagenin, sarsapogenin, tigogenin, yamogenin and yuccagenin.

However, the present invention relates more particularly to diosgenin. Diosgenin may be extracted from wild yam tubers by a process successively comprising: hydroysis of the heterosides in mineral acid medium (optionally after fermenting and drying the tubers); and filtration of the insoluble fraction, which is then neutralized, washed and treated with an apolar solvent. However, other extraction processes may be used. Diosgenin is also commercially available from the company Sigma under the trade name Diosgenin.

The expression “natural extracts” means any plant extract containing one or more sapogenins, after an optional treatment intended to hydrolyze the saponosides, such as an extract of wild yam rhizome, which contains diosgenin, or an extract of agave leaf containing hecogenin and tigogenin, or an extract of Liliacea plants, in particular of the genus Smilax or Yucca, containing smilagenin and sarsapogenin, such as an extract of sarsaparilla root.

The natural extracts used according to the invention may be obtained from plant material derived from a whole plant or part of a plant. This extract may be prepared from any plant material cultured in vivo or in vitro. The expression “in vivo culturing” means any culturing of conventional type, that is to say in the soil, in the open air or in a greenhouse, or alternatively out of the soil. The expression “in vitro culturing” means the combination of techniques known to those skilled in the art which makes it possible to artificially obtain a plant or a part of a plant. The selection pressure imposed by the physicochemical conditions during the growth of plant cells in vitro makes it possible to obtain a standardized plant material which is available throughout the year, in contrast with plants cultivated in vivo.

According to the invention, a natural extract derived from at least one plant of the genus Dioscorea is preferentially used, preferentially obtained from wild yam rhizome.

The genus Dioscorea belongs to the Dioscoreacea family and consists of hundreds of species, only a few of which contain sapogenins. Among these, examples that may be mentioned include Dioscorea villosa, Dioscorea opposita, Dioscorea composita, Dioscorea mexicana, Dioscorea tokora and Dioscorea floribunda.

The extract of the invention is preferentially prepared from plant material derived from the species Dioscorea villosa and/or Dioscorea opposita. Such an extract is especially available from the company Active Organics under the trade name Actigen Y®.

The amount of extract which may be used according to the invention obviously depends on the desired effect and may thus vary within a wide range.

To give an order of magnitude, the sapogenin, or the natural extract containing it, may be used in an amount representing from 0.0001% to 20% of the total weight of the composition, preferentially in an amount representing from 0.001% to 5% of the total weight of the composition and better still in an amount representing from 0.1% to 1% of the total weight of the composition.

The composition according to the invention is generally suitable for topical use and thus contains a physiologically acceptable medium, that is to say a medium that is compatible with the skin, its integuments and/or mucous membranes.

This composition may be in any presentation form normally used in cosmetics and dermatology, and it may especially be in the form of an optionally gelled aqueous solution, a dispersion of the lotion type, optionally a two-phase lotion, an emulsion obtained by dispersing a fatty phase in an aqueous phase (O/W emulsion) or conversely (W/O emulsion), or a triple emulsion (W/O/W or O/W/o emulsion) or a vesicular dispersion of ionic and/or non ionic type. These compositions are prepared according to the usual methods.

This composition may be more or less fluid and may have the appearance of a white or coloured cream, an ointment, a milk, a lotion, a serum, a paste or a mousse. It may optionally be applied to the skin in the form of an aerosol. It may also be in solid form, in particular in the form of a stick. It may be used as a care product, and/or as a makeup product for the skin.

In a known manner, the composition of the invention may contain adjuvants that are common in cosmetics, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the fields under consideration, and, for example, from 0.01% to 20% relative to the total weight of the composition. Depending on their nature, these adjuvants may be introduced into the fatty phase, into the aqueous phase, or into lipid vesicules. In any case, these adjuvants, and also the proportions thereof, will be chosen so not to harm the desired properties of the sapogenins or of the natural extracts according to the invention.

When the composition of the invention is an emulsion, the proportion of the fatty phase may range from 5% to 80% by weight and preferably from 5% to 50% by weight relative to the total weight of the composition. The oils, emulsifiers and co-emulsifiers used in the composition in emulsion form are chosen from those conventionally used in the field under consideration. The emulsifier and co-emulsifier are present in the composition in a proportion ranging from 0.3% to 30% by weight and preferably from 0.5% to 20% by weight relative to the total weight of the composition.

As oils which may be used in the invention, mention may be made of mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax or ozokerite) may also be used as fatty substances.

As examples of emulsifiers and co-emulsifiers that may be used in the invention, mention may be made of fatty acid esters of polyethylene glycol such as PEG-20 stearate, and fatty acid esters of glycerol such as glyceryl stearate.

Hydrophilic gelling agents that may be mentioned in particular include carboxyvinyl polymers (carbomer), acrylic copolymers such as acrylate/alkylacrylate copolymers, polyacrylamides, polysacchararides, natural gums and clays, and lipophilic gelling agents that may be mentioned include modified clays, for instance bentones, metal salts of fatty acids, hydrophobic silica and polyethylenes.

According to one preferred embodiment of the invention, the composition also contains at least one metalloprotease inhibitor, such as lycopene, isoflavones, derivatives thereof or plant extracts containing them, in particular extracts of soybean (sold, for example, by the company Ichimaru Pharcos under the trade name Flavosterone SB), of red clover, of flax, of kakkon or of sage. As a variant or in addition, the composition according to the invention may contain an agent for stimulating the growth and/or metabolism of fibroblasts, such as plant proteins or polypeptides, extracted especially by soybean (for example a soybean extract sold by the company LSN under the name Eleseryl SH-VEG 8 or sold by the company Silab under the trade name Raffermine), and/or an agent for promoting the differentiation and/or growth and/or metabolism of keratinocytes, such as retinol or its derivatives or certain plant extracts such as an extract of lupin sold by the company Silab under the trade name Structurine, and/or an agent for stimulating the cutaneous microcirculation, such as an extract of bitter orange sold by the company Silab under the trade name Remoduline.

The concentrations of these optional additional agents in the composition according to the invention can range from 0.0001 to 20% by weight and is preferably between 0.001 and 5% by weight relative to the total weight of the composition.

The composition according to the invention can also contain an agent for promoting collagen synthesis, such as ascorbic acid or its derivatives, an exfoliant such as α-hydroxy or β-hydroxy acids, a protective agent or UV screening agent, an agent for improving the skin barrier, such as ceramides, and/or a moisturizer such as polyols or calcium pantothenate.

Examples of UV screening agents that are particularly suitable for use in the present invention are:

butyl methoxydibenzoyl methane sold especially by the company Hoffmann-Laroche under the name Parsol 1789,

octocrylene sold especially by the company BASF under the name Uvinul N539,

octyl salicylate sold especially by the company Haarman-Reimer under the name Neo Heliopan OS,

octyl methoxycinnamate sold especially by the company Hoffmann-Laroche under the name Parsol MCX,

phenylbenzimidazole sulphonic acid sold especially by the company Merck under the name Eusolex 232,

oxybenzones such as benzophenone-3,-4, or -5,

benzotriazole silicones and in particular drometrizole trisiloxane,

terephthalylidene dicamphor sulphonic acid, and

titanium oxides or zinc oxides, in the form of optionally coated microparticles or nanoparticles.

The concentration of these UV screening agents in the composition according to the invention can range from 0.01% to 30% by weight and preferentially from 0.1 to 10% by weight relative to the total weight of the composition. These screening agents may be used alone or in combination to obtain the desired Sun Protection Factor in the UVA and/or UVB range.

The examples that follow are given for purely illustrative purposes and are not intended to limit the scope of the invention. Except where otherwise mentioned, the proportions referred to in the compositions below correspond to percentages by weight.

### EXAMPLE 1

### Effect of an Extract of Wild Yam on Collagenase Activity

The effect of an extract of wild yam on the inhibition of the activity of collagenase produced by Clostridium Histolyticum was evaluated. Although different from a human enzyme, this collagenase is used on account of its ability to digest human collagen. The positive reference is phenanthroline tested at concentrations of 1 mM, 0.5 mM and 0.25 mM in ethanol.

The extract of wild yam used is an extract of rhizome of the species Dioscorea Villosa sold by the company Active Organics under the trade name Actigen Y®, which was tested at concentrations of 2%, 1%, 0.5% and 0.1% (in a test buffer consisting of collagenase at 0.125 Units/ml, Tris at 0.1 M and CaCl₂ at 5 mM) of a stock solution at 2 mg/ml in 20/80 water/ethanol mixture, which corresponds to final concentrations of 40, 20, 10 and 2 μg/ml.

The control consisted of collagenase without test product. Thus, the activity of the control constitutes 100% collagenase activity and thus 0% inhibition of the said activity.

The products were preincubated with the enzyme for 10 minutes at room temperature, and DQ-gelatin was then added and the fluorescence obtained on degradation of the substrate was recorded for 10 minutes (excitation 480 nm; emission 538 nm) on a SpectraMax spectrofluorimetre (Molecular Devices); the results were processed using Soft Max Pro software.

The results obtained are collated in the table below. The percentages indicated represent the decrease in collagenase activity relative to the control.

| Treatment | % inhibition (n = 4) | p |
|---|---|---|
| Collagenase | 0 | — |
| Phenanthroline | | |
| 0.1 mM | 96 | <0.01 |
| 0.5 mM | 65 | <0.01 |
| 0.25 mM | 40 | <0.01 |
| Extract of wild yam | | |
| 40 μg/ml | 42 | <0.01 |
| 20 μg/ml | 39 | <0.01 |
| 10 μg/ml | 27 | <0.01 |
| 2 μg/ml | 22 | <0.01 |

The results show that the extract of wild yam he activity of the collagenase in a dose-dependent manner.

### EXAMPLE 2

### Emulsion in Cream Form

| | |
|---|---|
| Mixture of cetearyl alcohol and of cetearyl | 4% |
| glucoside | |
| Mixture of glyceryl stearate and PEG-100 | 1% |
| stearate | |
| Cetyl alcohol | 0.5% |
| C₁₂₋₁₅ alkyl benzoates | 2% |
| Hydrogenated polyisobutene | 5% |
| Petroleum jelly | 2% |
| Plant oils | 3.5% |
| Silicone wax | 2% |
| Volatile silicone | 5% |
| Extract of Padina pavonica | 1% |
| Extracts of wild yam | 0.7% |
| Isoflavone-rich extract of soybean | 1% |
| PEG-20 | 1% |
| Alcohol | 3% |
| Glycerol | 7% |
| Gelling agents | 1.5% |
| UVA and UVB screening agents | 12.5% |
| Neutralizing agent | 1.5% |
| Preserving agents | QS |
| Water | QS 100% |

This cream is used, in daily applications on the face and the neck, to prevent the signs of ageing of the skin and in particular loss of firmness of the skin.

1. Cosmetic use of at least one sapogenin or of a natural extract containing it to prevent the signs of ageing of the skin by inhibiting the activity of collagenases.

2. Cosmetic use of at least one sapogenin or of a natural extract containing it to prevent the loss of elasticity and/or tonicity of the skin and/or the formation of wrinkles and fine lines, by inhibiting the activity of collagenases.

3. Cosmetic use of at least one sapogenin or of a natural extract containing it to prevent the loss of firmness of the skin of the human face and/or neck.

4. Use according to any one of the preceding claims, characterized in that the sapogenin is chosen from diosgenin, hecogenin, smilagenin, sarsapogenin, tigogenin, yamogenin and yuccagenin.

5. Use according to claim 4, characterized in that the sapogenin is diosgenin.

6. Use according to any one of claims 1 to 3, characterized in that the natural extract is derived from at least one plant of the genus Dioscorea.

7. Use according to claim 6, characterized in that the natural extract is derived from at least one plant of the species Dioscorea villosa, Dioscorea opposita, Dioscorea composite, Dioscorea mexicana, Dioscorea tokora and Dioscorea floribunda.

8. Use according to claim 7, characterized in that the natural extract is derived from at least one plant of the species Dioscorea villosa and/or Dioscorea opposita.

9. Use according to any one of the preceding claims, characterized in that the sapogenin or the natural extract represents from 0.0001 to 20% of the total weight of the composition.

10. Use according to claim 9, characterized in that the sapogenin or the natural extract represents from 0.001% to 5% of the total weight of the composition.

11. Use according to claim 10, characterized in that the sapogenin or the natural extract represents from 0.1% to 1% of the total weight of the composition.
